(19) United States
(12) Patent Application Publication
(10) Pub. No.: US 2021/0209386 A1
(43) Pub. Date: Jul. 8, 2021

(54) RADAR-BASED MONITORING IN A VEHICLE

(71) Applicant: GENTEX CORPORATION, Zeeland, MI (US)

(72) Inventors: Matthew W. Remijn, Grand Rapids, MI (US); Benjamin N. Pohlman, Holland, MI (US); Brian G. Brackenbury, Grand Haven, MI (US); Joshua D. Lintz, Allendale, MI (US); Mark E. Ward, Grand Rapids, MI (US)

(73) Assignee: GENTEX CORPORATION, Zeeland, MI (US)

(21) Appl. No.: 17/140,813

(22) Filed: Jan. 4, 2021

Related U.S. Application Data
(60) Provisional application No. 62/956,376, filed on Jan. 2, 2020.

(51) Int. Cl. G06K 9/00; B60W 40/08

(52) U.S. Cl. CPC G06K 9/00838; B60W 40/08; B60W 2040/0881; B60W 2540/01; B60W 2420/52

(57) ABSTRACT

An occupant monitoring system in a vehicle comprises a processor in communication with a printed circuit board; a radar module in communication with the processor; and a lens in communication with the radar module. The radar module and the lens are disposed within a rearview assembly in a vehicle.

### CROSS REFERENCE TO RELATED APPLICATIONS

This application claims the benefit of and priority to U.S. Provisional Patent Application No. 62/956,376, filed on Jan. 2, 2020, entitled “Radar-Based Monitoring in a Vehicle,” the entire disclosure of which is hereby incorporated herein by reference.

### FIELD OF THE DISCLOSURE

This disclosure relates generally to monitoring of occupants in an interior of a vehicle, and in particular, to radar-based monitoring of occupants in an interior of a vehicle.

### SUMMARY

According to an aspect, an occupant monitoring system may comprise a processor in communication with a printed circuit board; a first radar module in communication with the processor; and a lens in communication with the first radar module. The first radar module and the lens may be disposed within a rearview assembly of a vehicle.

The lens may be configured to allow a field of view of the lens to capture at least one seating position for an occupant of the vehicle. The field of view of the lens may be configured to capture at least two occupant seating positions of the vehicle. The occupant monitoring system further may comprise a second radar module and a second lens in communication with the second radar module. The second radar module may be configured to be in communication with the processor. The second radar module and the second lens may be disposed in a ceiling of the vehicle. The second radar module and the second lens may be disposed in a ceiling console of the vehicle.

The first and second radar modules may be capable of detecting movements and collecting data related to at least one of lung function and heart function from at least one vehicle occupant. The processor may be configured to use the collected data to determine at least one of heart rate, pulse rate, heart rate variability, respiration rate, and heart rhythm. The processor may be configured to use the collected data to detect the location and presence of occupants of the vehicle. The occupant monitoring system may be configured to cause an alert to be generated if an occupant has been left in the vehicle after the occurrence of a predetermined event. The predetermined event may comprise, for example, at least one of the turning off of the vehicle, the locking of the vehicle doors from a location outside the vehicle, the passage of a predetermined amount of time, and the passing of a predetermined temperature threshold in the interior of the vehicle. The alert may comprise one of the vehicle horn sounding and an alert sent to a mobile phone. The radar module may comprise a 60 GHz FMCW radar. The radar module may comprise pulsed coherent radar. The occupant monitoring system may be configured to be in communication with other vehicle systems capable of causing the vehicle to be pulled off a road if a predetermined situation may be detected.

According to another aspect, a method of monitoring at least one physiological function of at least one occupant of a vehicle, may comprise: determining locations of occupants of the vehicle; gathering, using a radar module, data on at least one physiological function of at least one subject being monitored; and analyzing the collected data on a processor.

The subject being monitored may be an occupant of a vehicle. The radar module may be disposed in a rearview assembly of a vehicle. The method of monitoring further may comprise causing an alert to be transmitted in response to a determination that an occupant has been left in a vehicle and the occurrence of a predetermined event. The predetermined event may comprise at least one of the interior temperature of the vehicle reaching a predetermined threshold, the passage of a predetermined period of time, the locking of vehicle doors, a determination that the remaining occupant is in a particular seating position within the vehicle, and a determination that the remaining occupant is not in the driver position of the vehicle The subject being monitored may be the driver of the vehicle The method of monitoring further may comprise communicating, by a controller in communication with the processor and in response to a determination that the driver of the vehicle is impaired, with a vehicle system capable of causing the vehicle to pull off a road.

### BRIEF DESCRIPTION OF THE DRAWINGS

FIG. 1 illustrates a schematic diagram of a first embodiment of a radar-based monitoring system in accordance with this disclosure;

FIG. 2 illustrates a top view of a vehicle having the radar-based monitoring system of FIG. 1;

FIG. 3A illustrates a schematic diagram of a second embodiment of a radar-based monitoring system in accordance with this disclosure;

FIG. 3B illustrates a schematic diagram of a third embodiment of a radar-based monitoring system in accordance with this disclosure; and

FIG. 4 illustrates a flow chart of a method of utilizing the radar-based monitoring system of FIG. 1.

### DETAILED DESCRIPTION

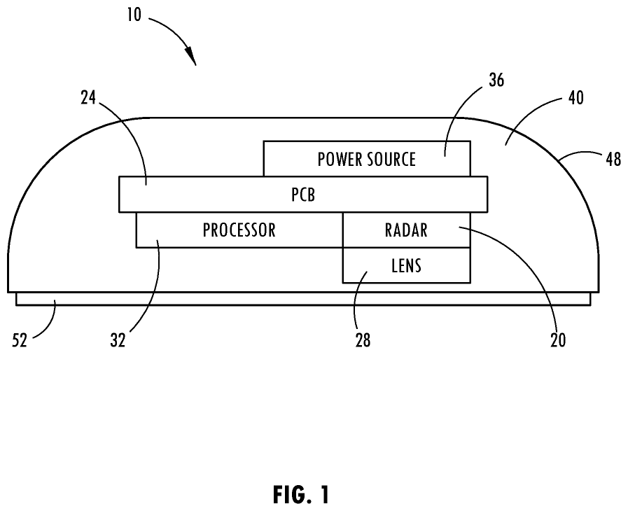

Referring to FIG. 1, a radar-based occupant monitoring system 10 may comprise at least one radar module 20 disposed on a printed circuit board 24. Each of the at least one radar modules 20 may comprise a lens 28. A processor 32 may be disposed on printed circuit board 24 and may be in communication with the at least one radar module 20. Radar-based occupant monitoring system 10 may receive power from vehicle power systems, or may have its own power source 36.

Radar-based occupant monitoring system 10 may be configured to convert received radar signals into digital data and to transmit them to processor 32 for analysis. It may be possible with radar-based occupant monitoring system 10 to detect lung and heart movements with high accuracy due, in part, to the penetrating abilities of the radar. Both cardiac and respiratory activity may cause a visible and measurable motion to the subject's chest wall. Thus, monitoring of particular physiological functions by radar-based occupant monitoring system 10 may be done through touch-free measurement of the vibrations of the vehicle occupant caused by the contraction of the heart muscle. Radar signals may reflect the diaphragmatic and chest wall movements.

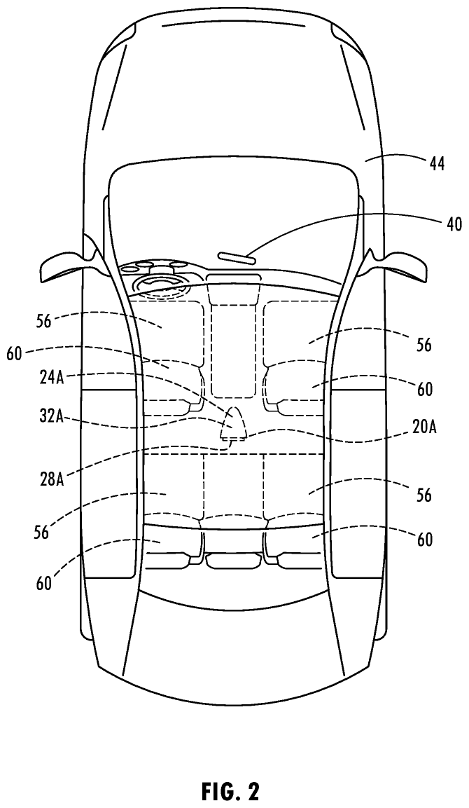

At least some components of radar-based occupant monitoring system 10 may be disposed in a rearview assembly 40 of a vehicle 44, as shown in FIG. 2. In some embodiments, lens 28 of radar-based occupant monitoring system 10 may be disposed in an edge or a housing 48 of rearview assembly 40. In some embodiments, lens 28 may be disposed in an interior cavity (not shown) of housing and capture data through a mirror, electrochromic, or display element 52 of rearview assembly 40. Lens 28 may have a field of view configured to capture at least one occupant seating position within vehicle 44 where the occupant seating position is a vehicle seat in which a vehicle occupant may sit. Occupant seating position may comprise, for example, the driver's seat or a passenger seat. Radar module 20 may be configured to capture information from vehicle occupants within a certain distance of radar module 20. Disposing radar module 20 in rearview assembly 40 may allow radar module 20 to be within the certain distance. In some embodiments, lens 28 may comprise a radar that is configured to determine azimuth through the use of beam forming through multiple transmit and receive antennas.

The data collected by radar module 20 may comprise information on at least one parameter such as the heart rate, pulse rate, heart rate variability, respiration rate, and heart rhythm of at least one vehicle occupant. The data may be processed by processor 32. The processing may include determining values for the heart rate, pulse rate, heart rate variability, respiration rate, and heart rhythm of occupants of vehicle 44. Processor 32 may be configured to process data and to provide almost instantaneous information on the chosen parameters, such as heartrates and heart rate variability. The data may also allow radar-based occupant monitoring system 10 to determine the location of occupants in vehicle 44. Additionally, radar-based occupant monitoring system 10 may be capable of monitoring multiple subjects simultaneously with one radar module 20, and/or of monitoring multiple measurement locations and/or multiple parameters on a single subject.

From a vantage point in the rearview assembly 40, radar-based occupant monitoring system 10 may be well-placed to monitor vehicle occupants from an unobstructed or generally unobstructed position. Being disposed in rearview assembly 40 may provide a field of view for lens 28 that allows it to capture data from multiple occupants of vehicle 44. Rearview assembly 40 may be at a more appropriate height than, for example a visor or roof of vehicle 44, as it is not as high and, thus, closer to vehicle occupants. Furthermore, in general, there are few or no obstructions between the rearview assembly 40 and the occupants in the front seats 56 of a vehicle 44. Due to its elevated and centralized location, rearview assembly 40 may also be well-positioned to monitor occupants in the rear seats of vehicle 44. Seat backs 60 of the front seats 56 may be low enough to allow radar module 20 in rearview assembly 40 to capture data from rear-seat occupants, and head rests are generally disposed far enough to the sides of vehicle 44 to allow signals from radar module 20 to travel between the headrests to gather information about rear-seat occupants. Furthermore, depending on the construction of vehicle seats 56 and head rests, radar signals may be capable of passing through the vehicle seats 56 and head rests.

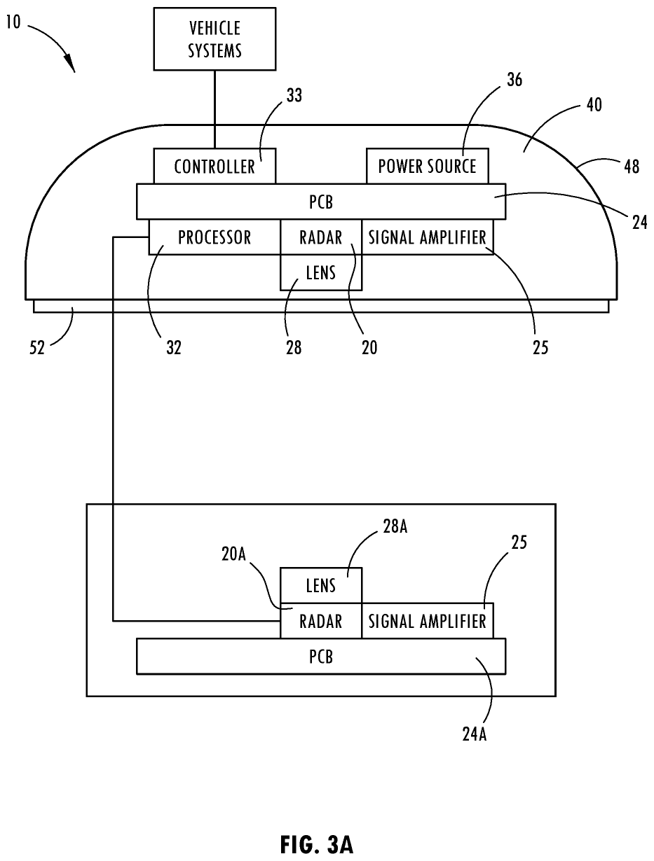

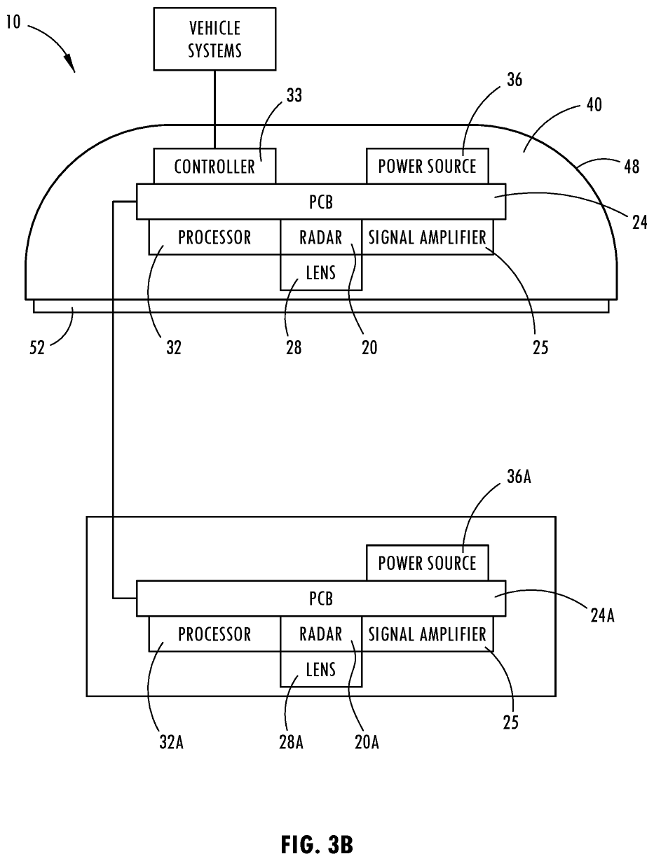

In vehicles 44 with more than two rows of seats 56, it may be possible for radar module 20 to capture data from occupants in the second or third rows of seats 56. However, it may be advantageous to install at least one additional radar module 20A and an additional lens 28A, as shown in FIGS. 3A and 3B, in a location within vehicle 44 closer to seating positions. Depending on the seating configuration within vehicle 44, at least one additional radar module 20A may be desirable. In particular, vehicles 44 having a third row of seats 56 may require an additional radar module 20A to monitor occupants of the third row of seats 56. In some embodiments, at least one additional radar module 20A may be required to allow monitoring of occupants in second row of seats 56, or in both second and third rows of seats 56. In some embodiments, a second radar module 20A may be used to monitor occupants in the second row of seats 56 and a third radar module (not shown) may be required to monitor occupants in the third row of seats 56. Each additional lens 28A may be in communication with one additional radar module 20A. In some embodiments, second and, if necessary, third radar modules 20A may be disposed in proximity to the second and third rows of seats 56, such as on a pillar of vehicle 44. In some embodiments, second and third radar modules 20A may be disposed in the ceiling of vehicle 44. In some embodiments, depending on distance of additional radar module 20A from occupant, signal amplification may be needed in order to collect the data.

The necessity for and number and location(s) of additional radar modules 20A may be determined based, in part, on the required or desired field of view, the configuration of vehicle 44, the width and field of view of lens 28 of radar module 20, and the sensitivity of radar module 20.

In some embodiments, a first signal amplifier 25 may be in communication with radar module 20. Signal amplifier 25 may be configured to amplify signals emitted by radar module 20. this may be necessary to increase the distance between radar module 20 and the vehicle occupant being monitored while maintaining effectiveness of the monitoring. A second signal amplifier 25A may be in communication with additional radar module 20A.

In some embodiments, as shown in FIG. 3A, any additional radar modules 20A, including second and third radar modules 20A if present, may be in communication with processor 32. Processor 32 may be in communication with printed circuit board 24. In some embodiments, additional radar modules 20A, including second and third radar modules 20A if present, may each be associated with an additional processor 32A, as shown in FIG. 3B. Each additional processor 32A may further be in communication with a subordinate printed circuit board 24A, each of which may be in communication with printed circuit board 24. In this arrangement, printed circuit board 24 may act as a master printed circuit board, being configured to communicate with subordinate printed circuit boards 24A and/or other vehicle systems. A controller 33 associated with printed circuit board 24 may be in communication with other vehicle systems such as those generating alerts such as activating the vehicle horn. Controller 33 may be configured to cause the generation and transmission of alerts by other vehicle systems when necessary based on data captured by at least one of radar module 20 and additional radar module(s) 20A.

Each additional radar module 20A may be configured to receive power from radar-based occupant monitoring system 10 or may be configured to receive power from vehicle power systems. In some embodiments, an optional power source 36A may be used to power additional radar modules 20A.

In some embodiments, additional radar module(s) 20A may be installed in a ceiling console of vehicle 44, such as a ceiling console having controls for video, audio, and/or ventilation systems. This may allow for a less obstructed view and/or better field of view for lens 28A of additional radar module 20A. In some embodiments, additional radar module(s) 20A may be disposed in the ceiling of vehicle 44.

In some embodiments, radar module 20 and additional radar module(s) 20A may comprise a first antenna (not shown) and a second antenna (not shown). Each antenna may comprise one or more antenna elements. Radar module(s) 20, 20A may be configured to transmit radar waves with the first antenna and to receive radar waves with the second antenna. In some embodiments, radar module(s) 20, 20A may further be configured to analyze the received radar waves.

In some embodiments, radar module(s) 20, 20A may be configured to detect, recognize, and analyze certain physiological functions of selected vehicle occupants. The monitoring of physiological functions may be non-invasive, non-contact monitoring. It may further be high precision and low cost, and use low power consumption. In some embodiments, the monitoring may be performed on both the driver and on vehicle passengers or on occupants of selected seating positions within vehicle 44. The certain physiological functions for which radar-based occupant monitoring system 10 is configured to capture information may include respiration rate, heart rate, heart rate variability, pulse rate, and heart rhythm.

In some embodiments, radar-based occupant monitoring system 10 may be configured to determine the locations of all occupants of a vehicle 44. Radar-based occupant monitoring system 10 may determine locations of occupants based on inputs received from lens(es) 28, 28A, from radar module(s) 20, 20A, or from inputs received from antennas. Radar-based occupant monitoring system 10 may be configured to receive inputs from each antenna or antenna element and use the inputs to determine the direction and distance of received signals to determine the locations of vehicle occupants.

In some embodiments, radar-based occupant monitoring system 10 may be configured to transmit an alert if a vehicle 44 is turned off and a passenger is left in vehicle 44. Alert may be generated, for example, if vehicle 44 is turned off and a heart rate or pulse is still detected within vehicle 44. In some embodiments, radar-based occupant monitoring system 10 may only cause an alert to be transmitted if an occupant is left in vehicle 44 and a predetermined event, such as the locking of vehicle 44, occurs. Other predetermined events may comprise the interior temperature of the vehicle reaching a predetermined threshold, the passage of a predetermined period of time, a determination that the remaining occupant is in a particular seating position within the vehicle, a determination that the remaining occupant is not in the driver position of the vehicle, and the like. For example, if an occupant is left in a vehicle 44 and a driver leaves and locks vehicle 44, radar-based occupant monitoring system 10 may be configured to cause an alert to be generated and transmitted. The alert may comprise, for example, at least one of sounding the horn of vehicle 44, or sending an alert to a mobile phone. Vehicle 44 may be configured to transmit the alert to a mobile phone that was recently paired to vehicle 44 through Bluetooth and/or to a mobile phone that has been entered into radar-based occupant monitoring system 10 as the mobile phone to receive notifications. Vehicle 44 may be configured to transmit the alert to all or a select subset of mobile phones that are listed in the vehicle directory as phones that pair with vehicle 44. Transmitting alerts in the event occupants are left in a vehicle 44 may prevent vehicle users from leaving children or infants in vehicle 44 after exiting vehicle 44.

In some embodiments, radar-based occupant monitoring system 10 may stay operational after vehicle 44 has been turned off. Radar-based occupant monitoring system 10 may be configured to operate using a back-up power source 36 such as a battery or to receive power from a vehicle system that remains active after vehicle 44 is turned off. In some embodiments, radar-based occupant monitoring system may be configured to receive power from the vehicle power systems while vehicle 44 is running, and to receive power from a back-up power source 36 when vehicle 44 is not running.

In some embodiments, radar-based occupant monitoring system 10 may be configured to monitor or track health of vehicle occupants. Radar-based occupant monitoring system 10 may be configured to be in communication with other vehicle systems, such as autonomous parking assistance or sensors that determine whether obstacles are in proximity to vehicle 44, and may be capable of causing vehicle 44 safely pull off a road if a hazardous situation is detected. The hazardous situation may include, for example, a drowsy driver or a driver suffering a cardiac event. Vehicle 44 may be pulled onto the shoulder of a road or off the road completely, for example, into a parking lot.

In some embodiments, radar module(s) 20, 20A may comprise pulsed coherent radar module(s). Pulsed coherent radar modules may be capable of detecting cardiac motions while located at some distance from the target. Pulsed coherent radar operates by transmitting signals to be reflected by obstacles and received by the radar. The received analog signals may be converted into digital data. The digital data may then be transmitted to processor 32 for further processing. Pulsed coherent radars typically have small sizes and simple structures, and are therefore generally easy to install and operate.

A pulsed coherent radar module may use pulsed coherent radar as a form of short-duration impulse waves, and the waves may enable the measurement of small motions with high accuracy, thus rendering the pulsed coherent radar module capable of detecting motions of the heart and lungs from outside the body while located a distance from the subject. This may allow radar-based occupant monitoring system 10 to be located at a greater distance (in some cases, up to several yards) from the subject than other non-contact vital sign monitoring methods. Due to its ultrawide bandwidth, the waves emitted by radar module 20 using pulsed coherent radar may penetrate clothing with minimal or no loss of accuracy.

Alternatively, in some embodiments, radar module 20 may comprise a frequency-modulated continuous wave (FMCW) radar module, such as a 60 GHZ FMCW radar, to monitor heart and breathing rates. The FMCW radar module may be configured to detect displacements caused by physiological movements, such as breathing and a heartbeat. Owing to the Doppler effect, the movements caused by the heartbeat and breathing can be detected by using a FMCW radar to transmit waves toward a subject and measuring phase shifts of a reflected radar signal, allowing the simultaneous measurement of heart rates and breathing rates.

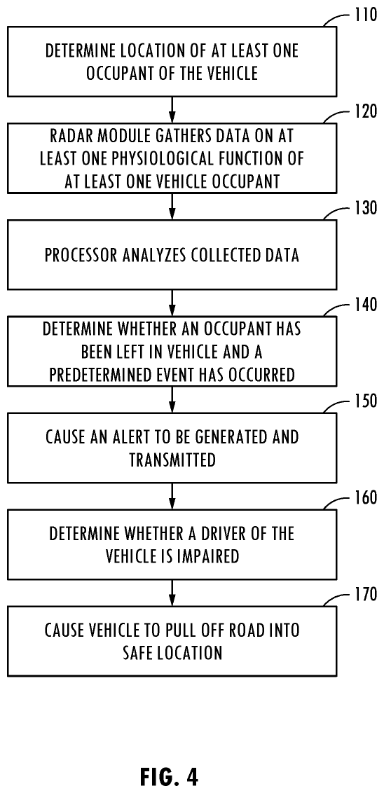

As shown in FIG. 4, a method of monitoring at least one occupant of a vehicle 44 is shown generally at 100. In step 110, a location of at least one occupant of vehicle 44 may be determined by radar-based occupant monitoring system 10. In step 120, radar module 20 may gather data, using radar waves, on at least one physiological function of at least one occupant of vehicle 44. In step 130, processor 32 may analyze the data collected by radar module 20.

In step 140, processor 32 may determine whether an occupant has been left in a vehicle and whether a predetermined event has occurred. In step 150, in response to the occurrence of a predetermined event and upon a determination an occupant has been left in vehicle 44, radar-based occupant monitoring system 10 may cause an alert to be generated and transmitted. The predetermined event may comprise at least one of the interior temperature of vehicle 44 reaching a predetermined threshold, the passage of a predetermined period of time, the locking of vehicle doors, a determination that the remaining occupant is in a particular seating position within vehicle 44, a determination that the remaining occupant is not in the driver position of vehicle 44, and the like.

In step 160, radar-based occupant monitoring system 10 may determine whether a driver of vehicle 44 is impaired. In step 170, in response to a determination that the driver of vehicle 44 is impaired, radar-based occupant monitoring system 10 may communicate with a vehicle system capable of causing vehicle 44 to pull off a road into a safe location. The safe location may be a shoulder of a road, a parking lot, and the like.

While the method was described using only one radar module 20 and one processor 32, it can be performed by a radar-based occupant monitoring system 10 using multiple radar modules 20A and multiple processors 32A.

The above description is considered that of the preferred embodiments only. Modifications of the disclosure will occur to those skilled in the art and to those who make or use the disclosure. Therefore, it is understood that the embodiments shown in the drawings and described above are merely for illustrative purposes and not intended to limit the scope of the disclosure, which is defined by the following claims as interpreted according to the principles of patent law, including the doctrine of equivalents. Although only a few embodiments of the present innovations have been described in detail in this disclosure, those skilled in the art who review this disclosure will readily appreciate that many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter recited. For example, elements shown as integrally formed may be constructed of multiple parts, or elements shown as multiple parts may be integrally formed, the operation of the interfaces may be reversed or otherwise varied, the length or width of the structures and/or members or connector or other elements of the system may be varied, the nature or number of adjustment positions provided between the elements may be varied. Accordingly, all such modifications are intended to be included within the scope of the present innovations. Other substitutions, modifications, changes, and omissions may be made in the design, operating conditions, and arrangement of the desired and other exemplary embodiments without departing from the spirit of the present innovations.

In this document, relational terms, such as first and second, top and bottom, front and back, left and right, vertical, horizontal, and the like, are used solely to distinguish one entity or action from another entity or action, without necessarily requiring or implying any actual such relationship, order, or number of such entities or actions. These terms are not meant to limit the element which they describe, as the various elements may be oriented differently in various applications. Furthermore, it is to be understood that the device may assume various orientations and step sequences, except where expressly specified to the contrary. It is also to be understood that the specific devices and processes illustrated in the attached drawings and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise.

It will be understood that any described processes or steps within described processes may be combined with other disclosed processes or steps to form structures within the scope of the present disclosure. The exemplary processes disclosed herein are for illustrative purposes and are not to be construed as limiting. It is also to be understood that variations and modifications can be made on the aforementioned methods without departing from the concepts of the present disclosure, and further it is to be understood that such concepts are intended to be covered by the following claims unless these claims by their language expressly state otherwise.

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point. The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within at least one of 2% of each other, 5% of each other, and 10% of each other.

1. An occupant monitoring system for a vehicle, comprising:
a processor in communication with a printed circuit board;
a first radar module in communication with the processor; and
a lens in communication with the first radar module;
wherein the first radar module and the lens are disposed within a rearview assembly in a vehicle.

2. The occupant monitoring system of claim 1, wherein the lens is configured to allow a field of view of the lens to capture at least one seating position for an occupant of the vehicle.

3. The occupant monitoring system of claim 2, wherein the field of view of the lens is configured to capture at least two occupant seating positions of the vehicle.

4. The occupant monitoring system of claim 1, further comprising:
a second radar module; and
a second lens in communication with the second radar module;
wherein the second radar module is configured to be in communication with the processor.

5. The occupant monitoring system of claim 4, wherein the second radar module and the second lens are disposed in a ceiling of the vehicle.

6. The occupant monitoring system of claim 1, wherein the first radar module is capable of detecting movements and collecting data related to at least one of lung function and heart function from at least one vehicle occupant.

7. The occupant monitoring system of claim 6, wherein the processor is configured to use the collected data to determine at least one of heart rate, pulse rate, heart rate variability, respiration rate, and heart rhythm.

8. The occupant monitoring system of claim 6, wherein the processor is configured to use the collected data to detect the presence and location of occupants of the vehicle.

9. The occupant monitoring system of claim 8, wherein the occupant monitoring system is configured to cause an alert to be generated if an occupant has been left in the vehicle after the occurrence of a predetermined event.

10. The occupant monitoring system of claim 9, wherein the alert comprises at least one of the vehicle horn sounding and an alert sent to a mobile phone.

11. The occupant monitoring system of claim 1, further comprising:
a second radar module;
a second lens in communication with the second radar module; and
a second processor in communication with the printed circuit board;
wherein the second radar module is configured to be in communication with the second processor.

12. The occupant monitoring system of claim 11, wherein the second radar module, the second lens, and the second processor are disposed in a ceiling of the vehicle.

13. The occupant monitoring system of claim 1, wherein the radar module comprises a 60 GHz FMCW radar.

14. The occupant monitoring system of claim 1, wherein the radar module comprises a pulsed coherent radar.

15. The occupant monitoring system of claim 1, wherein the occupant monitoring system is configured to be in communication with other vehicle systems and to cause the vehicle to remove itself from a road if a predetermined situation is detected.

16. A method of monitoring at least one physiological function of a vehicle occupant, comprising:
determining location of an occupant of the vehicle;
gathering, using a radar module, data on at least one physiological function of the occupant being monitored; and
analyzing the collected data on a processor.

17. The method of claim 16, wherein the subject being monitored is an occupant of a vehicle; and wherein the radar module is disposed in a rearview assembly of the vehicle.

18. The method of monitoring of claim 16, further comprising causing an alert to be transmitted in response to a determination that both:
an occupant has been left in a vehicle, and
the occurrence of a predetermined event.

19. The method of claim 18, wherein the predetermined event comprises at least one of the interior temperature of the vehicle reaching a predetermined threshold, the passage of a predetermined period of time, the locking of vehicle doors, a determination that the remaining occupant is in a particular seating position within the vehicle, and a determination that the remaining occupant is not in the driver position of the vehicle.

20. The method of monitoring of claim 16, wherein the subject being monitored is the driver of the vehicle; the method further comprising communicating, by a controller in communication with the processor and in response to a determination that the driver of the vehicle is impaired, with a vehicle system capable of causing the vehicle to pull off a road.
